(19) United States
(12) Patent Application Publication
(10) Pub. No.: US 2005/0038103 A1
(43) Pub. Date: Feb. 17, 2005

(54) Uses of dorzolamide

(76) Inventor: Amarjit Singh, Winnipeg (CA)

Correspondence Address: Dr. Michael R. Williams;Ade & Company, 1700-360 Main Street, Winnipeg, MB, R3C 3Z3, CA

(21) Appl. No.: 10/639,432

(22) Filed: Aug. 13, 2003

(51) Int. Cl. A61K 31/382

(52) U.S. Cl. 514/432

(57) ABSTRACT

A method of improving visual acuity and visual field in individuals in need of such treatment comprising administering to the eyes of an individual an effective amount of dorzolamide as well as a novel method of administering ocular medications is described.

### FIELD OF THE INVENTION

The present invention relates generally to the field of medical treatments. More specifically, the present invention relates to the use of dorzolamide to improve visual acuity and visual field in individuals in need of such treatment.

### SUMMARY OF THE INVENTION

According to a first aspect of the invention, there is provided a method of improving visual acuity or visual field comprising:
administering ocularly to an individual in need of such treatment an effective amount of dorzolamide.
According to a second aspect of the invention, there is provided a method of administering a medication to the eye comprising:
applying the medication to the eye of an individual in need of such treatment, and holding the eye of the individual open for a period such that the medication is absorbed by the eye.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a method of improving visual acuity in individuals in need of such treatment comprising administering to the eyes of said individual an effective amount of dorzolamide as well as a novel method of administering ocular medications.

Dorzolamide has been demonstrated to enhance contrast sensitivity and increase perimetric light sensitivity (Sponsel et al., 1997, Am J Ophthalmol 123: 759-766; Harris et al., 1999, J Ocular Pharmacology and Therapeutics 15: 189-197), enhance capillary dye transit in macula and optic nerve head (Harris et al., 1996, Acta Ophthalmologica Scandinavica 74: 569-572), accelerate retinal arteriovenous passage (Harris et al., 1999; Harris et al., 1996; Harris et al., 2001, Am J Ophthalmol 132: 490-495) and increase ocular pulse amplitude by 18-19% (Schmidt et al., 1998, Br J Ophthalmol 82: 758-762). However, dorzolamide has not previously been used to improve the visual acuity of individuals in need of such treatment.

Dorzolamide is available under the trade name TRUSOPT®) and is a known compound useful for the reduction of intraocular pressure as described in U.S. Pat. No. 4,797,413, which is incorporated herein by reference. As used herein, “dorzolamide” refers to dorzolamide as well as the pharmaceutically and ophthalmologically acceptable salts thereof.

Examples of individuals in need of such treatment include but are by no means limited to individuals in need of or desirous of improved night vision, for example, truck drivers, military personnel, pilots and the like, as well as individuals suffering from lensopacities, corneal dystrophies, age-related macular degeneration or retinitis pigmentosa.

It is of note that ocular medications have traditionally been administered by instructing the patient to apply one drop to the eye, then immediately close their eye and wipe any excess medication away. However, the act of closing the eye causes a film of the medication to be applied to the surface of the eye while the rest of the medication is expelled from the eye. As a consequence, a considerable portion of the medication is lost and wasted.

As noted above, there is provided herein a novel method of administering medication to the eye which provides more reproducible and consistent results, allows smaller volumes to be used and results in more medication being available to the eye for absorption and therefore better and more consistent effectiveness of the medication. As described in the examples below, the method involves applying a drop or less, for example, half a drop, of the medication to the open eye of the patient and thereafter keeping the eye open for a pre-determined period of time, in some embodiments, approximately 60 seconds, thereby providing more time for the medication to be absorbed by the eye, prior to closing the eye. Thus, in this method, very little or no medication is wasted. In an exemplary example, discussed below, the medicament administered by this method is dorzolamide. In other embodiments, other suitable medications are applied using this method, for example, but by no means limited to alphagan™, lumigan™, travatan™, azopt™, betoptic™, zalatan™, xalacom™, timolol™, betaxolol™, patanol™, maxidex™ and FML™.

Cataract refers to a clouding of the lens sufficient to reduce vision.

Age-related macular degeneration occurs in wet and dry forms. Here the drozolamide is used to improve visual acuity in the dry macular degeneration with retinal pigment epithelial changes of various degrees but not total absence of such retinal pigment epithelium (RPE) in the area of the macular.

Retinitis pigmentosa refers to a disparate group of rod and cone dystrophies characterized by progressive night blindness, visual field constriction and eventual loss of visual acuity and total blindness.

While not wishing to be bound to a particular theory, the inventor hypothesizes that dorzolamide improves circulation in the eye and that the increased blood flow in turn improves performance as measured by light sensitivity, contrast sensitivity, visual acuity and visual field.

It is of note that other suitable ophthalmological medicaments that improve circulation in the eye, for example, but by no means limited to, betaxolol, may be utilized instead of dorzolamide.

It is of note that in addition to improving visual acuity and visual field, administration of dorzolamide also improved the night vision of patients being treated for glaucoma. As such, it is clear that administration of dorzolamide would improve the night vision of individuals desirous of such treatment, for example but by no means limited to, truck drivers, military personnel, rescue personnel and the like. It is of note that in some embodiments, the dorzolamide may be administered in conjunction with the wearing of night vision goggles.

U.S. Pat. No. 6,046,223 claims the use of dorzolamide to treat or prevent macular edema and to prevent age-related macular degeneration, presumably by preventing macular edema from developing into age-related macular degeneration. However, this patent does not claim the treatment or improvement of visual acuity of individuals with established or confirmed age-related dry macular degeneration using dorzolamide.

The dorzolamide may be in the form of an ophthalmic pharmaceutical composition adapted for topical administration to the eye, such as, for example, but by no means limited to, a solution, ointment or solid insert.

The dorzolamide may be administered following manufacturer's directions. For example, the dorzolamide may be administered as one drop of a 2% solution twice daily. In other embodiments, the daily dosage may be 0.1 to 25 mg per day, either by single dose or on a 2 to 4 dose per day regimen.

In some embodiments, dorzolamide at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

The invention will now be described by way of examples; however, the invention is not in any way limited by the examples.

### EXAMPLE 1

Use of Dorzolamide in Improving Visual Acuity in Macular Degeneration

Patients with age-related macular degeneration (AMD) with drusens and macular pigment abnormalities were given Dorzolamide, gtts (i) bid in only one eye (i.e. better eye if both eyes were affected or the worse eye if the other was 20/20) for one month after which the visual acuity was reassessed. They were told that upon instilling gtts Dorzolamide they were to keep their eye open and count to 60 before wiping their eye. Improvement in visual acuity means that patients read one more line on Snellen's chart.

Most patients demonstrated objective (and subjective) improvement in visual acuity in one month which remained stable in some patients while in others there was continuing improvement in subsequent months. The results are summarized in Table 1.

Thus, Dorzolamide gtts (i) bid appears to be a very safe, simple, easy and quick way to improve visual acuity in many patients with AMD. It is of note that improved visual acuity and being able to “see better” was found to be a great morale booster in these patients who were resigned to gradually worsening vision with no hope of seeing better.

### EXAMPLE 2

Improvement in Visual Fields in a Patient with Retinitis Pigmentosa Following Dorzolamide treatment

A 23 year old healthy male investigated at the Ophthalmology department, Misericordia Health Centre, University of Manitoba, Winnipeg and the Eye Institute, Ottawa Hospital, Ottawa, with markedly constricted visual fields due to retinitis pigmentosa was given Dorzolamide, gtts (i) ou bid for five weeks after which the visual fields were reassessed. He was told that upon instilling gtts Dorzolamide he was to keep his eyes open and count to 60 before wiping his eyes. Gtts Dorzolamide was continued and visual fields were again reassessed after four weeks. The results are summarized in Table 2.

As can be seen in Table 2, the patient demonstrated marked improvement in visual field in the left eye in five weeks and still more marked improvement in both eyes in nine weeks of using gtts Dorzolamide.

Dorzolamide gtts (i) bid was thus found to be very safe, simple, easy and quick way to improve visual fields in this patient.

While the preferred embodiments of the invention have been described above, it will be recognized and understood that various modifications may be made therein, and the appended claims are intended to cover all such modifications which may fall within the spirit and scope of the invention.

1. A method of improving visual acuity comprising:
administering ocularly to an individual in need of such treatment an effective amount of dorzolamide.

2. The method according to claim 1 wherein the individual has age-related macular degeneration.

3. The method according to claim 1 wherein the individual has retinitis pigmentosa.

4. The method according to claim 1 wherein the individual has early-stage cataracts.

5. The method according to claim 1 wherein the individual has a corneal dystrophy.

6. The method according to claim 1 wherein the individual wants to see more clearly in a low light environment.

7. The method according to claim 1 wherein the dorzolamide is administered by applying the dorzolamide to the eye of the individual, and holding the eye of the individual open for a period of time so that the dorzolamide is better absorbed by the eye.

8. The method according to claim 7 wherein the period of time is at least about 60 seconds.

9. A method of administering a medication to the eye comprising:
applying the medication to the eye of an individual in need of such treatment, and holding the eye of the individual open for a period such that the medication is better absorbed by the eye and less medication is wasted.

10. The method according to claim 9 wherein the medication is applied as a half-drop and is just as effective as a full drop of medication.
